(19) United States
(12) Patent Application Publication
(10) Pub. No.: US 2026/0092218 A1
(43) Pub. Date: Apr. 2, 2026

(54) USY AND RELATED ZEOLITE CATALYSTS WITH BLOCKED UNDESIRED ACTIVE SITES, AND METHODS FOR SYNTHESIZING THE SAME RELYING ON SELECTIVE EXTRACTION AND ION-EXCHANGE

(71) Applicants: Chevron U.S.A. Inc., San Ramon, CA (US); The Regents of the University of California, Oakland, CA (US)

(72) Inventors: Alexander KATZ, Richmond, CA (US); Abraham MARTINEZ, Berkeley, CA (US); Le XU, Berkeley, CA (US); Jinyi HAN, San Ramon, CA (US); Alexander E. KUPERMAN, Richmond, CA (US); Cong-Yan CHEN, Kensington, CA (US); Bi-Zeng ZHAN, Albany, CA (US); Axel BRAIT, San Rafael, CA (US); Ram KUMAR, Berkeley, CA (US)

(73) Assignees: Chevron U.S.A. Inc., San Ramon, CA (US); The Regents of the University of California, Oakland, CA (US)

(21) Appl. No.: 19/090,967

(22) Filed: Mar. 26, 2025

Related U.S. Application Data
(60) Provisional application No. 63/701,105, filed on Sep. 30, 2024.

(51) Int. Cl. C10G 11/05; B01J 20/16; B01J 37/08; B01J 38/00; C07C 45/46

(52) U.S. Cl. CPC C10G 11/05; B01J 20/165; B01J 37/08; B01J 38/00; C07C 45/46

(57) ABSTRACT

Provided is a process for extracting organic surfactant occluded in an as-synthesized meso-Y zeolite (ASMY). The process comprises treating the ASMY with methanolic ammonium nitrate (MAN). The length of the treatment can last as long as needed, but equilibrium is generally reached at about 2 hours. The treatment is beneficial in that it does not decrease Al content and it extracts sufficient organic surfactant to be safe for calcination.

### CROSS REFERENCE TO RELATED APPLICATIONS

The present application claims priority to U.S. Provisional Application No. 63/701,105, filed Sep. 30, 2024, the complete disclosure of which is incorporated herein by reference in its entirety.

### BACKGROUND

### Field of the Disclosure

The present disclosure relates to the zeolite Y family of catalysts. More particularly, preparing mesoporous Y zeolites and removal of surfactant occluded in the pores of the mesoporous Y zeolites.

### Description of the Related Art

Zeolites are crystalline aluminosilicates, which are widely used solid catalysts in the production of fuels and chemicals. The zeolite Y family of catalysts (such as ultrastable Y (USY)) is based on the FAU framework and in particular has been implemented for decades in commercial processes such as fluid catalytic cracking (FCC), hydrocracking, and hydroisomerization. However, the nature of the active acid sites within these catalysts continues to be the subject of ongoing debate, especially related to the role of extra-framework A1 (EFA1) species that neighbor a small fraction (less than 20%) of framework A1 Brønsted acid sites and possibly render these sites to become more active. Current research efforts aim to better understand the role of highly active acid sites in the Y-family of zeolites, and also aim to synthesize hierarchical Y-family zeolites that include mesopores larger than 2.0 nm in diameter, which are referred to as meso-Y. These mesopores allow accessibility to sterically bulky reactant molecules, which would otherwise have difficulty diffusing through the narrow zeolitic portals (e.g., 0.74 nm). Such molecules are omnipresent in emerging feedstocks such as biomass, polymers (for their upcycling), as well as heavier fractions of crude oil-based hydrocarbons. One commercially implemented approach involves the dealumination of Y zeolites via steam and/or acid treatment, leading to USY zeolites (e.g., CBV-720, CBV-760, CBV-780).

In an effort to enlarge the mesopore volume of USY zeolites, a surfactant-templating process has been proposed involving one alkali-hydroxide treatment (alkali concentration typically in the range of 0.09 M to 0.2 M) of a USY zeolite (such as those mentioned above) with a long-chain quaternary ammonium cation surfactant such as cetyltrimethylammonium (CTA) bromide surfactant. This results in an as-synthesized USY zeolite that contains occluded CTA bromide organic surfactant in the amount of approximately 33 wt. %. This as-synthesized meso-Y zeolite is referred to as ASMY. Calcined ASMY is referred to as MY, and possesses a typical mesopore size of 4 nm and a mesopore volume of approximately 0.6 cm³/g (increasing alkali concentration during synthesis increases mesopore volume without significantly changing the mesopore size). The synthesis of an acid catalyst from ASMY is typically conducted by combusting the surfactant occluded within ASMY in a furnace (during the calcination alluded to above to synthesize MY), followed by ion exchanging the resulting MY material with aqueous ammonium nitrate solution, to yield a H-exchanged mesoporous Y (meso-Y) zeolite. Such a meso-Y zeolite synthesized according to such a conventional directly calcined approach is referred to as HMY.

An alternative approach aims to recycle some of the organic surfactant with an aqueous mineral-acid extraction solution, which sometimes also involves polar organic solvent components in the mixture such as acetone and ethanol. While such conditions are known in the art to lead to severe dealumination, the prospect of recycling some of the organic surfactant is advantageous from two perspectives: (i) it enables reuse of the organic surfactant, which is impossible in the combustion approach above, thereby giving cost savings as well as a more sustainable (environmentally friendly) approach; (ii) it allows the calcination of a smaller, residual amount (significantly smaller than the 33 wt. % of organic surfactant originally in the meso-Y material after synthesis) of organic surfactant occluded in the parent USY zeolite structure, which is advantageous when calcining to remove the final amount of surfactant after extraction, because it avoids the possibility of thermal runaway (i.e. a fire in the furnace) when a large wt. % of organic content is present.

Typically amounts of organic surfactant that are considered routine for combustion in zeolites have approximately 15 wt. % organic content (i.e. routinely encountered in the synthesis of zeolites), but roughly half of that in the ASMY. Improved methods of extracting surfactant from ASMY which avoid issues such as dealumination and combustion safety would be of value to the industry and would aid in preparing more active catalysts.

### SUMMARY

The present invention pertains to an alternative approach for extracting organic surfactant occluded in an ASMY. This approach is based on extraction of organic surfactant, and involves the use of methanolic ammonium nitrate (MAN) extraction of ASMY. A typical MAN concentration is 75 mM (milli moles) ammonium nitrate in methanol.

The present process for extracting an organic surfactant in an as-synthesized meso-Y zeolite (ASMY) comprise initially providing an as-synthesized meso-Y-zeolite (ASMY) that has been treated with an organic surfactant. The organic surfactant can be any suitable organic surfactant known for use in treating a meso-Y-zeolite. A preferred organic surfactant is a cetyltrimethylammonium (CTA) surfactant. The ASMY is treated with methanolic ammonium nitrate (MAN) for a suitable period of time and at a suitable temperature. The ASMY treated with the MAN is then recovered. The amount of remaining organic surfactant occluded in the zeolite after a single treatment is 18 wt. % or less, and generally 15 wt. % or less.

Among other factors, an advantage of the present inventive approach and process is that it synthesizes a catalyst in which a threshold level of organic surfactant is extracted, thereby rendering the resulting extracted meso-Y material safe from combustion because the wt. % of organic is in the range of about 15 wt. % or less, after a single equilibrium stage of extraction of ASMY with MAN. This also allows significant cost savings as over half of the occluded organic surfactant is extracted allowing for its recycling. An additional advantage of the present approach is that in contrast to previously described extraction approaches, the present extraction approach does not decrease Al content, which leads to the undesired removal of Brønsted acidity from the catalyst. A yet additional advantage of the present approach is that it also partially extracts occluded sodium cations in the ASMY, by exchanging these sodium cations with ammonium cations. The partial extraction of sodium cations is highly desirable from the standpoint of enabling the same level of Brønsted acidity as in conventional HMY meso-Y catalysts. However, due to the partial extraction of sodium cations, this leads to a catalyst with highly active sites still masked by a residual amount of sodium cations. It has been found that there is catalytic utility in having them remain masked for catalysis applications.

### BRIEF DESCRIPTION OF THE DRAWINGS

FIG. 1 shows a cetyltrimethylammonium (CTA) adsorption isotherm at 60° C. after equilibration with a methanolic ammonium nitrate solution (75 mM MAN). Black squares are experimental measurements and dotted line is experimental data fitted with a Freundlich adsorption isotherm. The top horizontal dashed line corresponds to total CTA in as-synthesized meso-Y zeolite (ASMY) and the lower dashed horizontal line is the CTA amount in ASMY after methanol Soxhlet extraction.

FIG. 2 shows a pie chart of cetyltrimethylammonium (CTA) distribution in as-synthesized meso-Y zeolite (ASMY).

FIG. 3 shows a sodium adsorption isotherm at 60° C. after equilibration with a methanolic ammonium nitrate solution (75 mM MAN). Black squares are experimental measurements and dotted line is experimental data fitted with a Freundlich adsorption isotherm. Top horizontal dashed line corresponds to total sodium in as-synthesized meso-Y zeolite (ASMY).

FIG. 4 shows a pie chart of sodium distribution in as-synthesized meso-Y zeolite (ASMY).

FIG. 5 shows the nitrogen physisorption isotherm measured at 77 K for various materials.

FIG. 6 shows the pore-size distribution calculated from the nitrogen physisorption isotherms in FIG. 5 (BJH method).

FIG. 7 shows the correlation between adsorbed sodium amounts and total Lewis acidity measured by pyridine IR spectroscopy.

FIG. 8 shows the nC₆ conversion during constraint index (CI) testing for H1EMY and HMY (left graph) and 3-MP conversion during CI testing for H1EMY and HMY (right graph). Filled black squares correspond to HMY and white open triangles correspond to H1EMY.

FIG. 9 shows the Friedel-Crafts acylation (FCA) equilibrium conversion of 2-methoxynaphthalene (2-MN) reactant as a function of stoichiometric excess of Ac₂O: 2-MN.

FIG. 10 shows the correlation between 1-acetyl-2-methoxynaphthalene (1,2-AMN) yield and total Lewis acidity measured by pyridine IR spectroscopy for materials in the EMY (extracted) series set.

FIG. 11 shows the correlation between 1-acetyl-2-methoxynaphthalene (1,2-AMN) yield and Na/Al ratio for materials related to the EMY (extracted) series set and CBV 720.

FIG. 12 shows the C₆ isomerization selectivity data from CI testing of H1EMY and HMY. Filled black squares correspond to HMY and white open triangles correspond to H1EMY.

FIG. 13 shows the 2-methylpentane (2-MP) yield data from constraint index (CI) testing of H1EMY and HMY. Filled black squares correspond to HMY and white open triangles correspond to H1EMY.

FIG. 14 shows the dimethylbutanes yield data from constraint index (CI) testing of H1EMY and HMY catalysts. Filled black squares correspond to HMY and white open triangles correspond to H1EMY.

FIG. 15 shows C₅ cracking selectivity data during constraint index (CI) testing for H1EMY and HMY. Filled black squares correspond to HMY and white open triangles correspond to H1EMY.

### DETAILED DESCRIPTION

Before the processes for extracting organic surfactants and preparing catalysts are disclosed and described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting. It must be noted that, as used in this specification, the singular forms “a,” “an,” and “the” include plural referents unless the context clearly dictates otherwise. Thus, for example, reference to “a step” may include multiple steps, reference to “producing” or “products” of a reaction or treatment should not be taken to be all of the products of a reaction/treatment, and reference to “treating” may include reference to one or more of such treatment steps. As such, the step of treating can include multiple or repeated treatment of similar materials/streams to produce identified treatment products.

Numerical values with “about” include typical experimental variances. As used herein, the term “about” means within a statistically meaningful range of a value, such as a stated particle size, concentration range, time frame, molecular weight, temperature, or pH. Such a range can be within an order of magnitude, typically within 10%, and more typically within 5% of the indicated value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every whole number integer within the range is also contemplated as an embodiment of the invention.

The present application relates to a process for extracting an organic surfactant from an as-synthesized meso-Y-zeolite (ASMY). The process comprises the use of methanolic ammonium nitrate (MAN).

The present process comprises initially providing an as-synthesized meso-Y-zeolite (ASMY) that has been treated with an organic surfactant. The organic surfactant is generally used to enlarge the mesopore volume of the Y zeolite. The organic surfactant can be any suitable organic surfactant known for use in treating a meso-Y-zeolite. A preferred organic surfactant is a cetyltrimethylammonium (CTA) surfactant. The ASMY is treated with methanolic ammonium nitrate (MAN) for a suitable period of time and at a suitable temperature. The ASMY treated with the MAN is then recovered.

The use of methanolic ammonium nitrate is of importance. The methanolic ammonium nitrate used in the treatment can comprise a suitable amount of ammonium nitrate in the ethanol. In one embodiment, the amount of ammonium nitrate in the methanol can range from about 25 to about 100 milli moles (mM) of ammonium nitrate. In another embodiment, the amount of ammonium nitrate in the methanol can range from about 50 to about 90 mM, or from about 70 to about 80 mM of ammonium nitrate in the methanol. Generally the MAN comprises about 75 mM of ammonium nitrate in methanol.

In treating the ASMY in the present process, the ratio of milligrams (mgs) of ASMY to milliliters (ml) of MAN used in the treatment can range from about 50:1 to about 10:1, or in one embodiment from about 40:1 to about 15:1. In another embodiment, the ratio of mgs of ASMY to ml of MAN in the treatment can range from about 30:1 to about 20:1, and preferably is about 20:1.

The length of time the ASMY is treated, in contact with the MAN, can be any suitable length of time. In one embodiment, the length of time can range from about 1 to about 4 hours, or from about 2 to about 4 hours. In one embodiment, the length of time of the treatment ranges from about 1.5 to about 2.5 hours, with about 2 hours often being suitable to reach equilibrium. The temperature during the treatment can be any suitable temperature, or sequence of temperatures. Generally maintaining the treatment at a single temperature or within a range of temperatures is sufficient. The treatment can be conducted at temperatures within the range of from about 50° C. to about 80° C., or in one embodiment in the temperature range of from about 55° C. to about 65° C. In one embodiment, the temperature of the treatment is about 60° C.

Therefore, in one embodiment, the present process can comprise treating an organic surfactant from an as-synthesized meso-Y-zeolite by treating the ASMY with the MAN for a length of time ranging from about 1 to 4 hours at a temperature in the range of about 50° C. to about 80° C. and with the MAN used in the treatment comprising from about 25 to about 100 mM of ammonium nitrate in methanol and the ratio of mgs of ASMY to ml of MAN in the treatment ranging from about 50:1 to 10:1. In another embodiment, for example, the process can comprise treating the ASMY with the MAN for a length of time ranging from about 1.5 to about 2.5 hours at a temperature in the range of from about 55° C. to about 65° C., and with the MAN used in the treatment comprising from 70 to 80 mM of ammonium nitrate in methanol, or in one embodiment about 75 mM of ammonium nitrate in methanol, and the ratio of mgs of ASMY to ml of MAN in the treatment of ranging from about 30:1 to about 20:1, and in another embodiment about 20:1.

A major advantage of the present process is that the process of extraction is efficient in extracting the organic surfactant to a level that is safe from combustion because the wt. % of organic surfactant, and hence organics, present after a single equilibrium stage of extraction of the ASMY with MAN is at such a safe level. It has been found that the amount of organic surfactant remaining in the recovered ASMY after treatment with MAN is generally at least about 18 wt. % or less, after a single treatment. The amount of organic surfactant can even be at about 15 wt. % or less. If further reduction in organic surfactant is desired, for example to 15 wt. %, 12 wt. % or less, a second extraction or even further extractions can be used.

Another benefit of the present process is that by removing so much of the occluded organic surfactant, the organic surfactant recovered can be recycled. This amounts to a significant cost savings as the organic surfactant can be expensive. The occluded surfactant can be any suitable organic surfactant that is used in treating a USY zeolite to enlarge the mesopore volume. Such surfactant templating processes are known. One significant organic surfactant is a long chain quaternary ammonium cation surfactant. In one embodiment, the organic surfactant is a cetyltrimethylammonium (CTA) surfactant, such as a bromide CTA surfactant. Being able to recover and recycle such a surfactant would offer significant cost savings.

From a catalyst standpoint, the present process offers further advantages. One additional advantage is that in contrast to previously described extraction approaches, the present extraction process does not decrease the aluminum (Al) content. Removal of the Al leads to the undesired removal of Brønsted acidity from the catalyst, so not decreasing the Al content is a major benefit. Furthermore, it has been discovered that the present process also partially extracts occluded sodium cations in the ASMY by exchanging sodium cations with ammonium cations during the treatment with MAN. Only partial extraction of sodium cations has been discovered to be highly desirable from the standpoint of enabling the same level of Brønsted acidity as in conventional meso-Y-catalysts. However, it has been found that due to only partial extraction of the sodium cations, a catalyst is obtained with highly active sites still masked by a residual amount of sodium cations. It has been found that there is catalytic utility in having them remain masked for catalytic applications.

Catalysts are prepared by calcining the recovered ASMY treated with the MAN. The calcination is conducted at conventional temperatures and conventional periods of time. For example, the temperature can range from about 500° C. to about 800° C., and in one embodiment from about 500° C. to about 700° C., for example about 580° C. The temperature can be controlled as is conventional, and can include ramping of the temperature to the final calcination temperature. The resulting catalysts can be useful in fluid catalytic cracking (FCC), hydrocracking and hydroisomerization reactions. The present particular catalysts obtained, comprising a portion of active sites masked by sodium cations, have also been found of particular usefulness in Friedel-Crafts acylation reactions.

The following examples are provided to further illustrate the present process and its utility and advantages. The examples are not meant to be limiting.

### EXAMPLES

### Example A (Comparative)

### Synthesis of Meso-Y Zeolite According to Literature Precedent

Literature precedent was followed for the synthesis of meso-Y zeolite according to the following procedure;

3.0 g of cetyltrimethylammonium (CTA) bromide were introduced into a 120 mL solution of 0.16 M sodium hydroxide in deionized water in a plastic (high-density polyethylene) round-bottom flask. This flask was heated in an oil bath at a temperature of 90° C. for 25 min as a pretreatment step. Following pretreatment, 6.0 g of parent USY material CBV 720 was introduced to the hot alkali surfactant solution with stirring of the resulting heated dispersion (at least 300 RPM) for 6 h. The dispersion was filtered with 6 L of 18.2 MΩ deionized water and was allowed to dry in a 60° C. oven overnight. The as-synthesized meso-Y zeolite is referred to as ASMY.

Conventional meso-Y was synthesized by first removing the surfactant contained within ASMY by combustion. This was performed by calcining ASMY in flowing air at 580° C. for 5 h (1 h ramp to 120° C. with 1 h isotherm at that temperature with a 5 h ramp to 580° C. with a 5 h isotherm; 250 mL/min of dry air per gram of material). This directly calcined ASMY material is referred to as MY. This same calcination procedure is followed elsewhere below where calcinations are referred to.

### Example 1

### CTA Extraction Approach and Results

CTA was extracted from ASMY and the extent of extraction was characterized with thermogravimetric analysis in air, resulting in a CTA coverage (in μmol/g) that is provided below on a basis of per gram of material at 750° C. (i.e. bone-dry zeolite material). Reported adsorbed CTA density is corrected for simultaneous ammonium exchange for both the extracted ion-exchangeable CTA cations and sodium cations. Effective molecular weights of adsorbed CTA species distinguish between physisorbed CTA corresponding to CTA (bromide/hydroxide, with the ratio of bromide to hydroxide reflecting the ratio in the synthesis mixture) and ion-exchangeable CTA cations corresponding to CTA without an extractable anion. This is discussed further below.

Data pertaining to extraction of all materials is summarized in Table 1 below. A typical extraction procedure involved treating 600 mg of ASMY in 30 mL of 75 mM MAN at 60° C. The resulting material was then filtered with water and dried overnight in a 60° C. oven. When this extraction was conducted for both 2 h and 4 h of duration, the same 53% of CTA was extracted from ASMY (ASMY had an initial CTA content of 1209 μmol/g), leading to a final adsorbed CTA concentration of 547 μmol CTA/g and a final CTA solution concentration of 13.2 mM. The same outcome for the 2 h and 4 h extraction experiments led to a conclusion that after 2 h of extraction the system is equilibrated. The resulting material is referred to as a once-extracted meso-Y material (1EMY). This material could be calcined to synthesize the H-form of 1EMY, which is referred to as H1EMY.

TABLE 1
Cetyltrimethylammonium (CTA) and sodium extraction data pertaining to different extracted
materials using methanolic ammonium nitrate (MAN), and their Al densities.

| | | | | CTA Sol | | Na Sol | |
|---|---|---|---|---|---|---|---|
| | | % CTA | CTA | Concentration | Na | Concentration | Al |
| Zeolite | % CTA | Extracted | (μmol/g) | (mM) | (μmol/g) | (mM) | (μmol/g) |
| ASMY | 35.7 | NA | 1209 | NA | 208 | NA | 1034 |
| 1EMY | 16.8 | 53.1 | 547 | 13.2 | 99 | 2.2 | 1051 |
| 2EMY | 11.9 | 66.7 | 441 | 2.1 | 53 | 0.9 | 1030 |
| ECEC | 0.0 | 100 | 0.0 | NA | 17 | NA | 1060 |

When 1EMY was extracted a second time (after 12 h of drying 1EMY at 60° C.) according to the same procedure as in the paragraph above, the residual CTA remaining in the twice-extracted material (heretofore referred to as 2EMY) was 441 μmol CTA/g, and the CTA solution concentration was 2.1 mM. It was concluded that an additional 20% of the CTA in 1EMY was extracted in a second equilibrium stage of extraction with MAN. Material 2EMY could be calcined to synthesize the H form of 2EMY, which is referred to as H2EMY.

ASMY was also extracted with a solution of MAN that initially consisted of 75 mM ammonium nitrate and 13 mM CTA bromide in methanol. This led to an extracted material with a final adsorbed CTA content of 625 μmol/g and a final CTA solution concentration of 24.9 mM. It was concluded that only 47% of the adsorbed CTA in ASMY was extracted in this modified procedure, which is significantly smaller than the fraction extracted during the synthesis of 1EMY above.

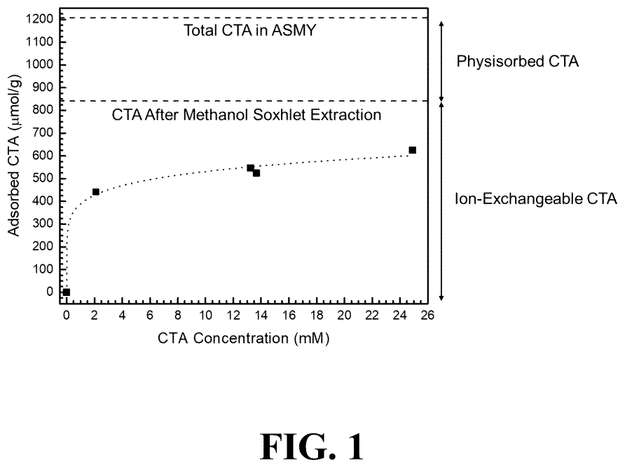

FIG. 1 of the Drawing shows CTA isotherm data of the materials described above, and a fit of these data to a Freundlich isotherm model. This model assumes a high degree of heterogeneity in the binding sites, by having sites with the highest heat of adsorption being populated at the lowest solution concentrations (in fact the distribution in the number of Freundlich adsorption sites having a heat of adsorption Q scales like exp (−Q/(R*n)) where R and n are constants). Thus, as the adsorbed amount increases (concomitantly as the solution concentration in the isotherm data increases) in the Freundlich isotherm model, the heat of adsorption decreases directly, thus accounting for the high degree of heterogeneity alluded to above. The good degree of fit of the present data in FIG. 1 to a Freundlich isotherm allows one to conclude that the bonding environments of CTA in ASMY are highly heterogeneous.

To probe these environments further, it was attempted to extract ASMY with methanol in a single equilibrium stage. This was conducted in the same fashion as above, except instead of using MAN, neat methanol was used. A final adsorbed CTA content of 972 μmol/g was observed in the methanol-extracted ASMY (corresponding to a CTA solution concentration of 2.7 mM). It was concluded that extraction with neat methanol removed 20% of the CTA in ASMY, and that this approach was less effective than extraction of ASMY with MAN above. The lower effectiveness of neat-methanol extraction might be rationalized on the basis of a lack of ionic strength in the extraction solution, which leads to only extraction of physiosorbed CTA. This physiosorbed CTA is paired with its own extractable anion (e.g., a statistical mixture of bromide and hydroxide that is representative of the synthesis solution during meso-Y synthesis) and is to be differentiated from ion-exchangeable CTA, which is paired with a tetrahedral MO₄⁻ anion (wherein M is either Al or Si) in the zeolite (vide supra).

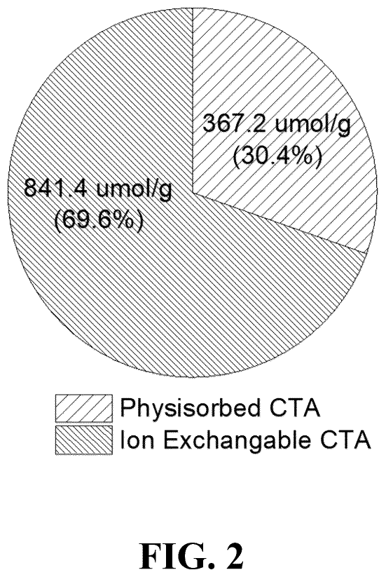

The data above further detail the heterogeneity of CTA environments in ASMY, and these data are summarized in the pie chart of FIG. 2 as well as the dashed horizontal lines of FIG. 1. In particular, in addition to heterogeneity in the ion-exchangeable CTA described above for ASMY, a significant (30%) population of physisorbed CTA within ASMY was also observed.

### Example 2

### Sodium Exchange Results

The adsorbed sodium amounts for ASMY, 1EMY, and 2EMY were measured via ICP analysis, and calculated the corresponding final sodium extraction-solution concentrations in MAN for 1EMY and 2EMY. All adsorbed sodium densities were normalized on a per gram of material at 750° C. (i.e. bone-dry zeolite material) basis. These sodium extraction data are shown in Table 1. These data correspond to adsorbed sodium amounts of 208 μmol/g sodium for ASMY, 99 μmol/g for 1EMY (final sodium extraction-solution concentration of 2.2 mM), and 53 μmol/g 2EMY (final sodium extraction-solution concentration of 0.91 mM) in MAN. When ASMY extraction was conducted for both 2 h and 4 h durations in MAN, the same amount of sodium extraction was observed, leading to a conclusion that after 2 h of extraction the system was equilibrated.

ASMY was also extracted with a solution of MAN that initially consisted of both 75 mM ammonium nitrate and 2.2 mM sodium nitrate in methanol. This led to an extracted material with a final adsorbed sodium content of 104 μmol/g (final sodium extraction-solution concentration of 4.3 mM in MAN). It was concluded that twice the final sodium extraction-solution concentration in MAN led to nearly the same adsorbed sodium amount, when comparing 1EMY to the material extracted in the presence of added sodium nitrate in MAN described above.

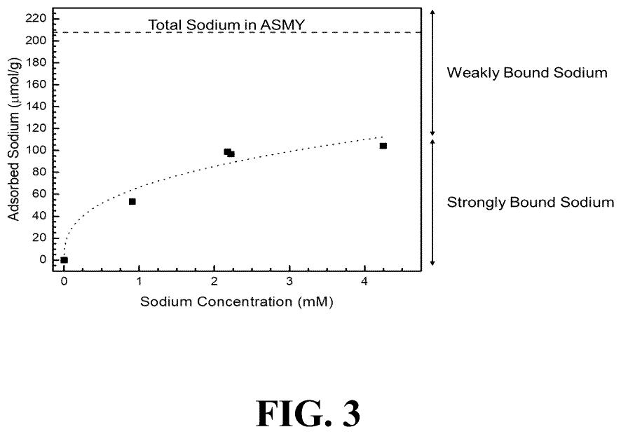

FIG. 3 shows a sodium adsorption isotherm of the materials described above, and a fit of these data to a Freundlich isotherm model. The good degree of fit of the data in FIG. 1 to a Freundlich isotherm allowed a conclusion that the bonding environments of sodium in ASMY is highly heterogeneous.

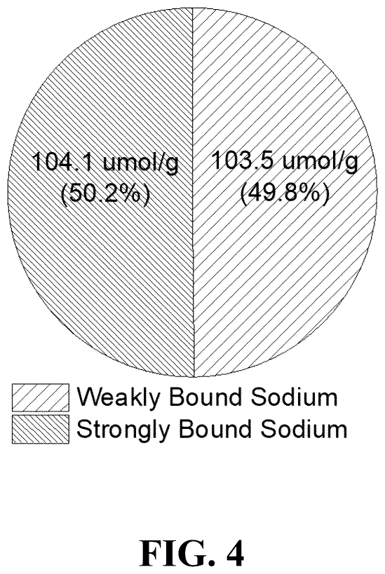

The sodium isotherm data above further detail the heterogeneity of sodium environments in ASMY, and these data are summarized in the pie chart of FIG. 4 as well as the dashed horizontal lines of FIG. 3. Just like CTA had two highly different regimes for adsorption (i.e., physiosorbed CTA and ion-exchangeable CTA), sodium also has two different regimes in FIGS. 3 and 4, consisting of weakly-bound and strongly-bound sodium adsorption sites. In contrast to results with CTA, however, Soxhlet extraction of ASMY in methanol led to a material with the same adsorbed sodium amount as in ASMY. It was concluded that all sodium was bound to an unextractable aluminosilicate anion (e.g., tetrahedral A104).

It was also observed that the summation of total sodium cations and ion-exchangeable CTA was nearly identical to the total aluminum sites within the material. This is evidenced in Table 1, which shows that the aluminum density for all materials discussed thus far was approximately 1.05 mmol/g. It was concluded that the present CTA extraction procedure does not destroy Al sites within the EMY set of materials, wherein EMY refers to materials derived from ASMY that have been extracted one or more times in MAN.

The observed mechanisms of cationic adsorption on FAU-based zeolites contrast with previously reported sodium isotherms in USY materials, which have been reported to follow a Langmuir isotherm. These prior data imply that each sodium adsorption site is identical and has the same affinity towards sodium, suggesting each site consists of a uniform distribution of tetrahedral A104-bonding environments. This evidence supports prior data that show similar deprotonation energies for tetrahedral A104 anionic sites in Y zeolites. These prior data emphasize the surprising nature of the present findings relating to the highly heterogeneous subpopulation of sodium adsorption sites (which follow a Freundlich isotherm as described above) in meso-Y materials.

### Example 3

### Competitive Adsorption Between CTA and Sodium Cations

To assess the degree of competitive adsorption of CTA and sodium cations, ASMY was extracted with MAN solutions that separately consisted of increased levels of CTA and sodium. One such solution consisted of twice the amount of CTA (i.e. the final CTA concentration in MAN after extraction was 24.9 mM instead of 13.2 mM for 1EMY above, while the final sodium concentration in MAN after extraction was nearly the same as for 1EMY at 2.2 mM). Surprisingly, it was found that the adsorbed sodium amount of ASMY in equilibrium with this new MAN solution having nearly double the CTA concentration was 97 μmol/g, which is nearly the same as the 99 μmol/g described above for 1EMY. This is further surprising in view of the CTA concentration in MAN being up to 6.2-fold higher than the sodium concentration and yet having no effect on the sodium adsorption in the data described above. It was concluded that sodium adsorption does not occur competitively with CTA at sodium adsorption sites. This is surprising from the standpoint of both sodium and CTA being monovalently charged cations, which would be expected to compete for the same tetrahedral anionic sites based on charge considerations. The lack of observed competition between sodium and CTA for sodium adsorption sites within meso-Y might mean that the environment around the sodium adsorption site tailors its selectivity to sodium by not allowing CTA to adsorb.

More sodium was introduced to a MAN extraction solution and extracted ASMY with this new solution, which resulted in a final sodium concentration after extraction of 4.3 mM compared 2.2 mM for 1EMY above, while keeping the CTA concentration after extraction nearly the same as for 1EMY at 13.6 mM. Surprisingly, it was found that the adsorbed CTA amount of the meso-Y material in equilibrium with this new MAN solution having nearly double sodium concentration was 525 μmol CTA/g, which was nearly the same as the 547 μmol CTA/g described above for 1EMY. It was concluded that CTA adsorption does not occur competitively with sodium at CTA adsorption sites in ASMY. This is surprising for the same reasons described above for sodium adsorption sites being selective to sodium in the presence of excess CTA in MAN solution. These data require the environment around each CTA adsorption site to be tailored to CTA such that adsorption of sodium to these same sites does not occur in a competitive fashion.

The data above point to a high degree of selectivity for both CTA and sodium adsorption sites in ASMY, such that both of these sites operate in a non-competitive fashion for their respective cations. Further evidence of this selectivity is evident when considering the much higher ammonium cation concentration of 75 mM in MAN. If adsorption sites were operating in an unselective manner for monovalent cations, we would expect the much higher ammonium concentrations in MAN in all of the adsorption experiments to result in nearly no adsorption of both CTA as well as sodium cations from MAN, if adsorption occurred purely based on entropic effects. However, such a result is inconsistent with the curvature of observed isotherms in CTA and sodium concentration ranges that are much smaller than 75 mM in MAN in FIGS. 1 and 3, respectively. The data in these Figures require sites that operate selectively for their respective cations in the face of much higher ammonium cation concentrations in MAN. In conclusion, highly selective adsorption sites were observed for sodium and CTA in meso-Y as reflected by: (i) non-competitive adsorption of CTA in the presence of sodium; (ii) non-competitive adsorption of sodium in the presence of CTA; (iii) selective adsorption sites that are selective towards either sodium or CTA (based on (i) and (ii) above) and that simultaneously are nonselective towards the much higher (by up to 28-fold) final (i.e. after extraction) ammonium concentrations in MAN solution. These selective adsorption sites for sodium and CTA in meso-Y are also observed to be highly heterogeneous in nature, with a small fraction of these sites having the highest affinity towards sodium and CTA based on the adsorption data in FIGS. 1-4.

### Example 4

### Synthesis of Control Materials

In addition to the synthesis of 1EMY and 2EMY above consisting of materials containing varying amounts of CTA and sodium that were equilibrated with a MAN solution during extraction, a variant that had no remaining CTA content prior to equilibration with MAN was also synthesized. This was performed by calcining 1EMY, causing the removal of remaining CTA via combustion (i.e., to synthesize H1EMY). The resulting calcined material was re-extracted with a MAN solution (75 mM ammonium nitrate in methanol) under the same conditions described above for the EMY materials. This extraction caused ammonium exchange of residual adsorbed sodium cations in H1EMY, leading to material ECEC. The ECEC was calcined to synthesize the final calcined material HECEC following the above-mentioned extraction with MAN. Because the material being calcined in the final calcination step was ammonium exchanged, this led to proton Brønsted acid sites in HECEC after final calcination. HECEC had a low level of adsorbed sodium consisting of 17 μmol/g, which is the lowest level of adsorbed sodium measured for all MAN-extracted materials. It was rationalized that this low level of adsorbed sodium resulted by the opening up of the aluminosilicate network of meso-Y as a result of the steam created during surfactant combustion during the calcination of 1EMY, in the process of synthesizing HECEC.

A control material consisting of directly calcined ASMY, MY was also synthesized. This was performed by calcining ASMY as described in Example A. This intermediate calcined (e.g. MY) material was ion exchanged with aqueous ammonium nitrate (1.0 M) solution by treating 1 g of ASMY with 50 mL of this solution at 60° C. and repeating this exchange for a total of three times. The thrice ion exchanged material is defined as NH4MY. NH4MY was finally calcined to synthesize HMY consisting of a directly calcined material in the proton form. For reference, HMY contained 13 μmol/g of sodium, which was nearly identical to that of HECEC.

The extracted materials were compared with those synthesized according to a previously described CTA extraction approach, which involves using a ethanolic solution of mineral acid, as described in Boorse et al. (U.S. Pat. No. 9,963,349). The procedure implemented involved taking 600 mg of ASMY and extracting it with 150 mL of a 1.09 M HCl/EtOH (mineral acid ethanolic (MAE)) solution at reflux for two hours (oil bath temperature measured 92° C.). Upon extraction, the material was filtered, washed with water, and dried overnight in an oven at 60° C. The resulting material is referred to as REMY. TGA analysis of REMY showed 121 μmol CTA/g, consistent with extraction of greater than 90% of CTA from ASMY with this approach, as claimed in the original patent by Boorse et al. (U.S. Pat. No. 9,963,349). The resulting material was also highly dealuminated, as a result of the strong acid and will be discussed in further detail below.

### Example 5

### Nitrogen Physisorption

Nitrogen adsorption isotherms at 77 K were measured on a Micromeritics ASAP2020 adsorption instrument. The mesopore (external) surface area and micropore volumes were determined by the t-plot method and the mesopore volume determined by the Barrett-Joyner-Halenda (BJH) method. Prior to sample analysis, samples were degassed at 350° C. for 4 hours under vacuum to remove residual water species prior to measurement. All samples were calcined prior to measurement.

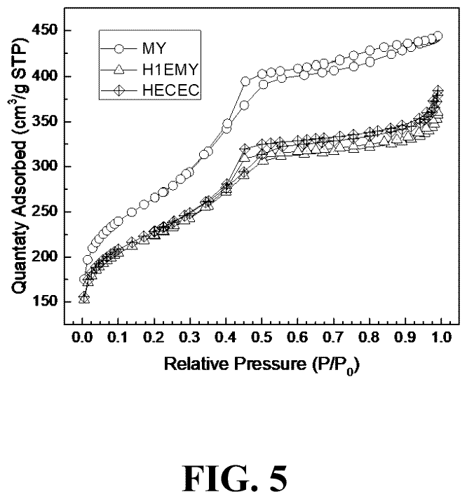

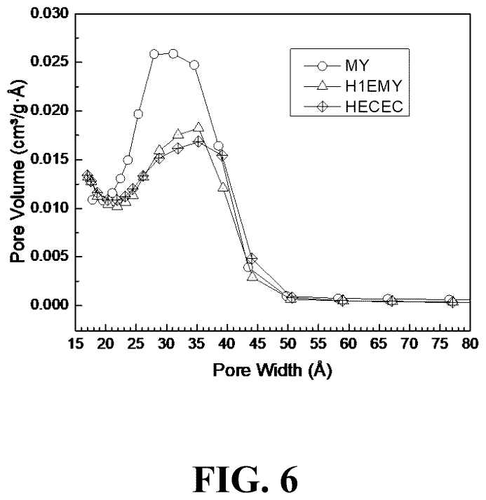

Nitrogen physisorption isotherms measured at 77 K for extracted EMY and control HMY are shown in FIG. 5, with the textural characteristics summarized in Table 2. The isotherms of all materials exhibit a type IV isotherm, commensurate with mesoporous silicates synthesized via micellar templating. It was observed that MY has a larger uptake of nitrogen at P/P₀>0.40 range, which is indicative of greater mesoporosity. This is corroborated by the pore size distribution shown in FIG. 6, which demonstrates MY to have the largest mesopore volume, while all materials have the same 4.0 nm mesopore diameter. Table 2 shows MY to have a mesopore volume of 0.58 cm³/g, which is larger than the mesopore volumes observed for H1EMY (0.45 cm³/g). It was surmised that the larger number of mesopores synthesized in MY was due to the larger amount of steam generated as a result of CTAB combustion during high temperature calcination. It was expected that more steam would be generated during calcination in the synthesis of MY compared to H1EMY due to the larger CTAB content of ASMY (1209 μmol CTA/g corresponding to 35.7 wt. %) compared with the H1EMY after extraction (547 μmol CTA/g corresponding to 16.8 wt. %). This steam synthesized mesopores at the expense of zeolitic micropores due to known high-temperature hydrolysis and dissolution processes involving the zeolite framework.

TABLE 2
Micropore and mesopore volumes derived from nitrogen
physisorption measured at 77K on various materials.

| | Micro | Meso |
|---|---|---|
| | PV | PV |
| Zeolite | (cm³/g) | (cm³/g) |
| CBV 720 | 0.26 | 0.24 |
| MY | 0.14 | 0.58 |
| H1EMY | 0.15 | 0.45 |
| H2EMY | 0.15 | 0.46 |
| HECEC | 0.15 | 0.49 |
| HMY | 0.15 | 0.62 |

However, data in Table 2 demonstrate a similar mesopore volume for all calcined materials in the extracted-material series involving H1EMY, H2EMY, and HECEC, in the range of 0.45-0.49 cm³/g. In addition, within this same extracted-material series, all materials have the same micropore volume of 0.15 cm³/g. It was concluded that within this extracted-material series, differences in the amount of CTA combustion during calcination did not cause appreciable zeolite hydrolysis and dissolution, as it does for ASMY. This is an advantage to using the extraction approach developed in this invention, in addition to the previously discussed aspect of decreasing the risk of thermal runaway (fire) during calcination, as a direct result of the lower CTA contents in the extracted materials relative to ASM.

### Example 6

### Pyridine Desorption Measured Via FTIR Spectroscopy

The number of Brønsted and Lewis acid sites for all materials of interest was quantified with pyridine FTIR spectroscopy on moisture-free per-gram basis. Self-supported wafers of approximately 10 mg were loaded into a flow cell and treated under 10-7 torr vacuum at 673 K for two hours to dehydrate the sample prior to pyridine uptake. Background spectrum was hence collected under the vacuum with the presence of the wafer at 423 K. Following which, the self-supported wafer was exposed to three dosages of pyridine at 100 mTorr and subsequently 2 dosages of pyridine at 1 Torr. Absorption spectrum was collected 24 hours post-exposure under vacuum at 423 K for the determination of the concentration of total Lewis/Brønsted sites. Strong acid sites concentration was determined following raising the temperature of the wafer to 623 K and dwelling for 24 hours. Absorption spectrum was acquired after cooling the wafer back to 423 K. Integration of the Lewis and Brønsted bands corresponding to v (PyH+) at 1545 cm⁻¹ and v (PyL) at 1450 cm⁻¹ were used to determine the corresponding density of sites, using extinction coefficients determined in N. S. Nesterenko's work. All FTIR spectra were recorded using a Nicolet 6700 spectrometer with a spectral resolution of 4 cm⁻¹ and 400 scans.

Table 3 summarizes the data. An increase in the density of Brønsted acid sites (BAS) was observed when comparing the twice MAN-extracted H2EMY (260 μmol BAS/g) with the once extracted H1EMY (214 μmol BAS/g). Material HECEC (268 μmol BAS/g) had a similar Brønsted-acid site density compared with H2EMY. The Brønsted acid-site density for the directly calcined control HMY is 207 μmol BAS/g and nearly the same as for H1EMY.

TABLE 3
Elemental compositions from inductively coupled plasma - optical emission
spectroscopy (ICP-OES); total Brønsted acid site (TBAS) and total
Lewis acid site (TLAS) measured by pyridine IR spectroscopy; OH/OD density
measured by IR spectroscopy before (OH) and after (OD) exchange with
deuterated benzene; 1-acetyl-2-methoxynaphthalene (1,2-AMN) yield from
Friedel-Crafts acylation (FCA) catalysis with various materials.

| | | | | | | | 1,2- |
|---|---|---|---|---|---|---|---|
| | | | TBAS | TLAS | OH | OD | AMN |
| Zeolite | Si/Al | Na/Al | (μmol/g) | (μmol/g) | (μmol/g) | (μmol/g) | Yield |
| CBV 720 | 14.5 | 0.01 | 304 | 163 | 380 | 360 | 22.4 |
| ASMY | 12.3 | 0.20 | NA | NA | NA | NA | NA |
| H1EMY | 12.3 | 0.09 | 214 | 262 | 135 | 127 | 3.0 |
| H2EMY | 12.2 | 0.05 | 260 | 315 | 162 | 149 | 8.1 |
| HECEC | 12.3 | 0.02 | 268 | 362 | 102 | 100 | 15.8 |
| HMY | 13.4 | 0.01 | 207 | 281 | 130 | 120 | 15.2 |
| REMY | 39.7 | NA | 86 | 51 | 90 | 100 | 19.9 |

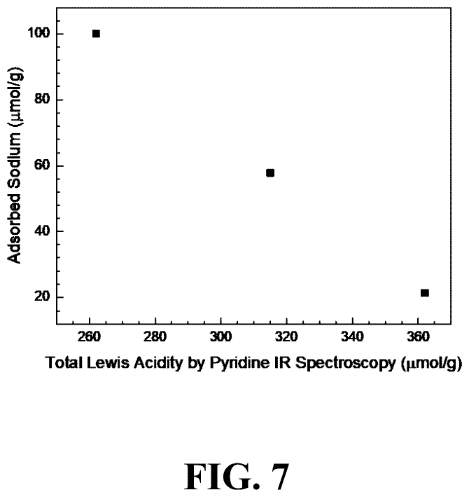

Trends for quantified Lewis acid site (LAS) densities in Table 3 also demonstrate increases when comparing H1EMY (262 μmol LAS/g) and H2EMY (315 μmol LAS/g). However, the latter LAS density was significantly lower relative to the value measured for HECEC (362 μmol LAS/g), demonstrating a continuing growth in LAS density with each extractive treatment, when progressing with H1EMY<H2EMY<HECEC, which was not observed for BAS densities above. Correlating these LAS density increases in the series of MAN-extracted materials above demonstrated LAS to be correlated with an uncovering of residual sodium adsorption sites via extraction. It was surmised that the environment corresponding to the most tightly held sodium adsorption sites is highly Lewis acidic (accounting for an increase in LAS between HECEC and H2EMY) rather than Brønsted acidic (accounting for nearly the same BAS in HECEC and H2EMY). The relationship between adsorbed sodium cations extracted and total LAS density is shown graphically in FIG. 7, and is observed to be linear. The LAS density in HMY was measured to be 281 μmol LAS/g, which was slightly larger than observed for H1EMY above.

Also quantified was the number of Brønsted and Lewis acid sites for REMY, an extracted variant prepared according to a previously described CTA extraction approach, which involves using a ethanolic solution of mineral acid, as described in Boorse et al. (U.S. Pat. No. 9,963,349). The pyridine number densities are reported in Table 3 and the results show a Brønsted acid site density of 86 μmol BAS/g. This value was drastically reduced compared to the directly calcined and ion-exchanged HMY (i.e. 207 μmol BAS/g), as well as the disclosed HEMY series of materials (268 μmol BAS/g). Results in Table 3 also demonstrated measured Lewis acid sites density of 51 μmol LAS/g for REMY. This value was also drastically reduced in comparison to all other materials measured. The total Al site density was measured to be 294 μmol/g, which is significantly smaller than the approximate 1.05 mmol/g. It was concluded that these decreased acid site densities results from the dealumination of the catalysts during the extraction process to synthesize REMY.

### Example 7

### OH/OD Exchanges Measured Via FTIR Spectroscopy

The number of exchangeable Brønsted-acidic OH sites was quantified using deuterated benzene exchange (C₆D₆) measured via FTIR spectroscopy, and Table 3 summarizes these data. The concentration of acid sites was calculated on moisture-free per-gram basis. OH/OD exchange was performed by loading self-supported wafers of approximately 10 mg into a flow cell and treating under 10-7 Torr vacuum at 673 K for two hours to dehydrate the sample prior to exposure to C₆D₆. Exchange took place by exposing the dry zeolite to 5 Torr of C₆D₆ at 353 K for 30 s with repeated pulses until no changes in spectral peak intensities are observed in both OH (3700-3450 cm⁻¹) and OD (2750-2550 cm⁻¹) spectral windows. Integration of the peak areas was used for determination of OH/OD exchanges using Emeis' reported extinction coefficients (C. A. Emeis, J. Catal. 1993, 141 (2), 347-354).

An increase in the OD density (integrated from the entire OD spectral window) was observed when comparing the twice MAN-extracted H2EMY (149 μmol OD/g) with the once extracted H1EMY (127 μmol OD/g). The OD density for the directly calcined control HMY was 120 μmol OD/g, which was nearly the same as for H1EMY. It was observed that OH integration densities values are within ˜10% of OD integration densities and followed these same trends described here above. It was concluded that the overall trends in OH/OD exchange were consistent with trend in Brønsted acid site density measured by pyridine IR spectroscopy above. In particular, the Brønsted acid-site density for 1HEMY and HMY were nearly identical, whereas H2EMY had a slightly (about 20%) higher density of Brønsted (and Lewis) acid sites compared with H1EMY.

The number of exchangeable Brønsted-acidic OH sites for control material REMY was quantified using the approach above and are reported in Table 3. The results showed an OD density of 100 μmol/g. This value was similar in magnitude compared to the directly calcined and ion-exchanged HMY, as well as the disclosed HEMY series of materials. This contrasts the pyridine acid site densities, as previously measured significant decreased levels in both BAS and LAS densities for REMY. These OH/OD acid site densities were apparently unaffected by dealumination REMY experienced during synthesis.

### Example 8

### Constraint Index (CI) Testing: Cracking Activity

Constraint index (CI) testing was performed in order to measure the activity of present catalysts for reactions that are known to be catalyzed by Brønsted acid sites, involving cracking reactions of n-hexane (nC₆) and 3-methypentane (3-MP) of these C₆ isomers.

Calcined variants consisting of H1EMY and HMY were pelletized at 4-5 KPSI and crushed and meshed to 20-40 size. Then, 0.47 g of material (dry weight as determined by TGA at 600° C.) was packed into a ⅜″ stainless steel tube with catalytically inactive alundum on both sides of the zeolite bed. An ATS (Applied Test Systems, Inc.) furnace was used to heat the reactor tube. Helium was introduced into the reactor at 23 ml/min and atmospheric pressure. The catalyst was dehydrated at 482° C. for 2 hours. The temperature was then reduced to 427° C. and helium flow was adjusted to 9.4 mL/min and an equimolar mixture feed of n-hexane (nC₆) and 3-methylpentane (3-MP) was introduced into the reactor at a rate of 0.48 mL/h. The feed delivery was made via an ISCO pump. The on-line sampling of the product into a gas chromatograph (GC) began after 15 min of feed introduction. The CI value (including 2-methylpentane formation) was calculated from the GC data using methods known in the art.

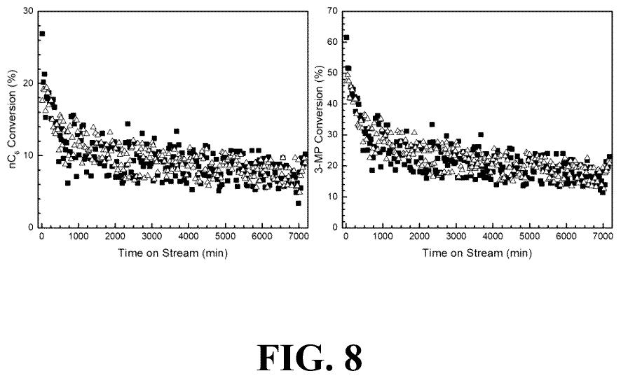

FIG. 8 shows the nC₆ and 3-MP conversions comparing HMY and H1EMY for up to 120 h time on stream. Linear data extrapolated to zero time on stream (taken from data during the first 360 min) showed an initial nC₆ conversion of 20.6% and 19.9% for H1EMY and HMY, respectively. These data also demonstrated an initial 3-MP conversion of 51.2% and 51.0% for H1EMY and HMY, respectively. Both HMY and H1EMY exhibit similar catalyzed reaction rates for the cracking of both reactants. This seemed to suggest (i) the acid sites within both H1EMY and HMY (both have FAU framework topologies) are equally highly accessible to both reactants and (ii) HMY and H1EMY have similar Brønsted acid site densities of 210.5±3.5 μmol). At longer times on stream (>360 min), the 3-MP and nC₆ conversion data in FIG. 8 show very similar values for both catalysts as well, consistent with the conclusions and interpretation described above.

### Example 9

### Low Temperature Friedel-Crafts Acylation of 2-Methoxynaphthalene

Friedel-Crafts acylation (FCA) of 2-methoxynaphthalene (2-MN) with acetic anhydride (Ac₂O) was conducted as an acid-catalyzed probe reaction under extraordinarily mild conditions of 40° C. The goal was to use the activity of a given catalyst in catalyzing this reaction under such mild conditions as a surrogate for the presence of highly active Brønsted acidic sites within that catalyst. Previously, in the literature, this reaction was shown to be catalyzed by Brønsted acid sites of zeolites (in their H-exchanged form). However, previously reported FCA reactions with zeolite catalysts have been typically conducted at elevated temperatures of greater than 60° C.

The FCA catalysis experiments were conducted under extraordinarily mild conditions as follows. Reagents 1,2-dichloroethane (DCE) and Ac₂O were dried first under calcium hydride for at least a week and were subsequently dried in a solid mixture of phosphorous pentoxide/potassium carbonate. They were subsequently distilled and stored under nitrogen atmosphere. Catalysts were calcined in a tube furnace and transferred to a moisture-free argon-filled glovebox prior to use. Solid 2-MN was used as received in an argon-filled glovebox. In a typical reaction, 100 mg of zeolite and 1.1 mmol of 2-MN were loaded into separate Schlenk tubes and were sealed to maintain a moisture free environment. The tubes were placed under a nitrogen atmosphere, and 10 mL of DCE, 0.105-1.05 mL Ac₂O (˜1.1-11.1 mmol) and 250 μL of dodecane were added in an air-free manner via syringe to the tube containing the 2-MN. This range of Ac₂O to 2-MN controls the molar ratio of these two reactants to be between 1 and 10. It was found a molar ratio of Ac₂O to 2-MN of 3 was ideal for initial rate measurements that were far from the equilibrium conversion with the procedure below. The catalyst and reagent tube were allowed to preheat up to the reaction temperature of 40° C. for 15 minutes, before the reagent solution was introduced via cannula transfer into the preheated catalyst. The reaction was typically conducted for a period of 10 minutes, after which the reaction mixture was filtered with a 0.2 μm PTFE filter. The filtered reaction mixture was analyzed on an Agilent 6890 GC system equipped with an FID and HP-1 column for chemical analysis. For all reactions, both the carbon balance and the mole balance were nearly closed to within greater than 90% for the former and greater than 95% for the latter. The major product formed was 1-acetyl-2-methoxynaphthalene (1,2-AMN) at greater than 97% selectivity for all experiments.

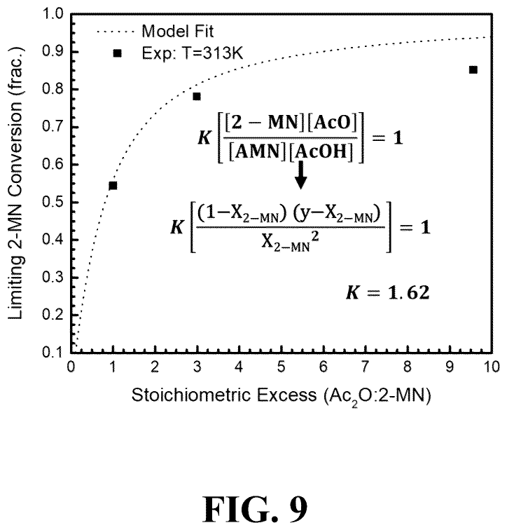

The mild conditions as controlled by the low reaction temperature of 40° C. allowed a measurement of an equilibrium constant for this reaction by measuring equilibrium conversions at different stoichiometric ratios of Ac₂O to 2-MN. Results shown in FIG. 9 demonstrate an equilibrium constant of 1.62 measured at 40° C. Such a measurement of the equilibrium constant was possible because of the extraordinarily mild conditions, which allowed one to investigate this reaction at initial rate regimes while largely avoiding catalyst deactivation that occurs at higher temperatures.

Table 3 shows the 1,2-AMN product yield for the set of calcined extracted EMY materials as well as control HMY. An increase in the product yield was observed when comparing H2EMY (8.1% 1, 2-AMN yield) with H1EMY (3.0% 1, 2-AMN yield), showing that an additional stage of extraction synthesizes a more active FCA catalyst under the present conditions. Material HECEC had a significantly larger 1,2-AMN product yield of 15.4%, though it is noted in passing that its Brønsted acid site density was found to be similar to H2EMY as described above. The activity of HECEC was similar to the 15.8% 1,2-AMN product yield achieved in the directly calcined control HMY catalyst.

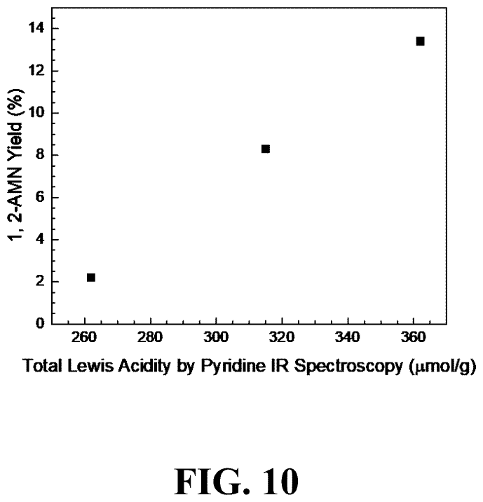

The observed increases in 1,2-AMN product yield were correlated with the total Lewis acid sites in the set of extracted EMY materials, and the direct nature of this relationship is shown in FIG. 10. It was thought that the strong acid sites responsible for low-temperature FCA activity under the present conditions involved total Lewis-acid sites, a residual amount of which are uncovered during sodium extraction in MAN. When sodium cations are adsorbed prior to their extraction, they must block a fraction of total Lewis-acid sites, rendering the resulting catalyst to be less active for our FCA reaction. In summary, extraction removed sodium ions according to the isotherm data in FIG. 3, which as described above, uncovered a residual amount of total Lewis acid sites, which play an active role in participating in the FCA catalysis under our mild conditions.

### Example 10

### Low Temperature Friedel-Crafts Acylation of Methoxybenzene

To ensure that the effects above were not the result of unexpected exclusion of reactants and products from the active site, a low-temperature FCA catalysis was conducted on a smaller aromatic reactant, methoxybenzene (MB), under otherwise the same mild FCA reaction conditions as described above. Data in Table 4 demonstrates that when replacing 2-MN with MB under the same mild FCA reaction conditions, the yield of 4-methoxyacetophenone (4-MOAP) product was 1.4% and 4.4% for H1EMY and HECEC, respectively. It was concluded that H1EMY is about 3 times slower compared to HECEC. Because this factor was close to the factor of 5 previously observed when comparing these catalysts under mild FCA reaction conditions with 2-MN as the reactant, it was concluded that the observed differences in the catalyst activity were independent of reactant size and did not reflect a shape selective effect. It was concluded that these differences must reflect the presence and absence of highly active sites in HECEC and H1EMY, respectively.

TABLE 4
Friedel-Crafts acylation (FCA) catalysis data representing
product yields of 1-acetyl-2-methoxynaphthalene (1,2-
AMN) and 4-methoxyacetophenone (4-MOAP), along with
the Na/Al ratio of each catalyst investigated.

| | | 1,2-AMN | 4-MOAP |
|---|---|---|---|
| Zeolite | Na/Al | Yield (%) | Yield (%) |
| 1EMY | 0.09 | 3.0 | 1.40 |
| HECEC | 0.02 | 15.4 | 4.40 |

### Example 11

### Low Temperature Friedel-Crafts Acylation of 2-Methoxynaphthalene: Sodium Reintroduction and Role of Sodium

To further test the hypothesis of sodium cations uncovering residual Lewis acid sites as being the critical controlling variable for FCA catalysis, sodium cations were reintroduced to both NH4MY (recall NH4MY is defined as thrice ammonium ion-exchanged MY prior to a final calcination) and ECEC in a targeted fashion. The reintroduction of sodium was performed by adding 20 mL of aqueous sodium bicarbonate (the targeted amount of sodium was reached by adjusting the aqueous solution concentration to be in the range of 0.60-1.72 mM) to 400 mg of catalyst in its ammonium cation exchanged form i.e. before its final calcination (e.g., ECEC not HECEC). This treatment was performed at room temperature for 24 h under stirring. The material was subsequently filtered with 1 L of deionized water and dried overnight in a 60° C. oven. The targeted amount of sodium reintroduction as controlled by the sodium bicarbonate amount in solution was transferred to the solid catalyst quantitatively in nearly 100% yield with the procedure above.

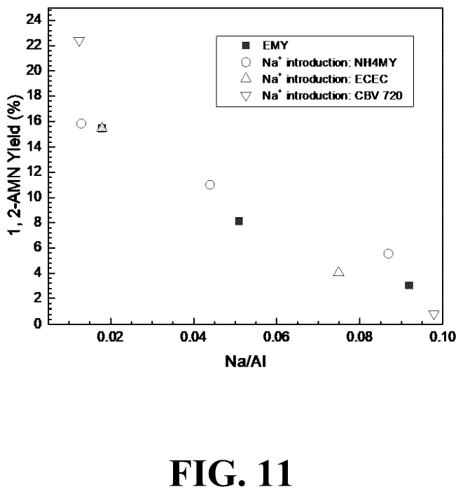

The results shown in FIG. 11 demonstrate FCA activity for all materials, including those in which sodium was reintroduced, as a function of the sodium to aluminum ratio in the catalyst. A direct correlation was observed between this ratio and the FCA activity for both HMY and HECEC catalysts, as well as the entire lack of observed FCA activity for all catalysts at a sodium to aluminum ratio of larger than approximately 0.1. It is noted that all of the aluminum in calculating this ratio is ion exchangeable with either CTA cations or sodium cations (vide supra), and is therefore differentiated from total aluminum in other materials, in which a portion of the aluminum is not ion exchangeable. In those instances, it has been argued that only the ion-exchangeable fraction of aluminum is the relevant one. The observed direct relationship between residual sodium levels (ratioed to aluminum content) and FCA catalytic activity in such a broad class of materials in FIG. 11 supports the uncovering of a small subset of acid sites as being required for mild FCA catalytic activity. Together with the data in FIG. 10, it was concluded that a small subset of Lewis acid sites are required for our mild FCA activity. This selectivity was manifested by the small sodium to aluminum ratios above (less than 10%), knowing that sodium titrates at least 20% of total aluminum in FIG. 11. The data in FIG. 11 suggests that sodium preferentially titrates the strongest Lewis acid sites in the distribution first-those that are responsible for FCA catalytic activity under our conditions.

### Example 12

### Generality of Low Temperature Friedel-Crafts Acylation of 2-Methoxynaphthalene

To examine the generality of the observations outside of HMY and the set of MAN-extracted materials, FCA catalysis under the conditions with previously known control USY catalysts was conducted. These include REMY, which is an extracted variant of ASMY, which was extracted with concentrated mineral acid according to U.S. Pat. No. 9,963,349 (vide supra), and other USY based materials such as CBV 720 (Si/Al=14.5, the parent material from which our mesoporous Y syntheses began from) and a more dealuminated variant of a HUSY catalyst CBV 760 (Si/Al=30, a material similar to CBV 720 that has been dealuminated more severely with a combination of steam and acid treatment). All of these materials were calcined prior to FCA catalysis. Catalytic results shown in Table 3 and FIG. 11 demonstrate 1,2-AMN product yields of 20.4% and 22.4% for REMY and CBV 720, respectively. It is noted that CBV 760 exhibited a 1,2-AMN product yield of 30.3%. This suggests that all of these known materials exhibit FCA catalytic activity, in stark contrast to H1EMY (recall the 1,2-AMN product yield under the same conditions was 3% for H1EMY with Si/Al ratio of 12.3). This observation differentiates the material of this invention, H1EMY, from the remaining materials known a priori. The result is surprising because many of the materials above that are active for FCA catalysis under the present conditions possess much lower aluminum and acid-site density than 1EMY (e.g., vide supra for a discussion of this pertaining to REMY).

When sodium was reintroduced to CBV 720 with the same approach as described above for HMY and HECEC, data in FIG. 11 demonstrate the complete deactivation of FCA catalytic activity under the present conditions, represented by a 1,2-AMN yield of 0.8%, at a sodium to aluminum ratio of approximately 0.1. These results demonstrate the generality of high-activity sites present in USY zeolitic materials that are outside of the meso-Y family (i.e. meso-Y family refers to alkaline-treated USY materials in the presence of a long-chain surfactant such as CTA bromide). It was concluded from these results that there is a minor population of highly active catalytic sites within all of the materials investigated that is responsible for FCA catalytic activity under our conditions.

An additional investigation was conducted to rule out burst kinetics as being responsible for the FCA test results. Burst kinetics refers to catalysis results that show a burst of activity in the beginning but rapidly deactivate thereafter. To demonstrate the lack of burst kinetics, the amount of catalyst was halved and the reaction time doubled. Due to rapid deactivation under burst kinetics, if such kinetics are operative, then the yield will be lower than in the case of a full amount of catalyst at a normal (not doubled) reaction time. Data in Table 5 with CBV 720 demonstrates the same 1,2-AMN yield in both of the cases described above. It seems the low-temperature FCA results were not consistent with burst kinetics and represented stable catalytic sites within the active catalysts described above.

TABLE 5
Friedel-Crafts Acylation (FCA) batch reactor data
with different CBV 720 catalyst loadings.

| Catalyst | Reaction | 1,2-AMN |
|---|---|---|
| Wt. | Time | Yield |
| (mg) | (min) | (%) |
| 100 | 10 | 22.4 |
| 50 | 20 | 21.8 |

To further demonstrate the uniqueness of these highly active catalytic sites within the context of other zeolitic materials that are outside of the USY family, FCA catalysis was performed under the present conditions on various pure-phase large-pore and medium-pore aluminosilicate zeolites, as well as control alumina and aluminosilicate materials. The data shown in Table 6 demonstrate that none of these materials exhibited a 1,2-AMN yield above 1.5% AMN under the conditions. It was concluded that acid sites found in the zeolites and aluminosilicates of Table 6 were not sufficiently active for FCA catalysis under the present conditions.

TABLE 6
Friedel-Crafts Acylation (FCA) catalysis data from other
pure phase zeolites and aluminosilicates as catalysts.

| | | 1,2-AMN |
|---|---|---|
| Catalyst | Si/Al | Yield (%) |
| (H)Al-SSZ70 | 50 | 0.7 |
| (H)SSZ-60 | NA | 0.0 |
| (H)MOR | 10 | 1.1 |
| (H)ZSM-5 | 25 | 0.0 |
| (H)BEA-CP814E | 12.5 | 0.6 |
| (H)Al-MCM-41 | 15 | 2.7 |
| γ-Al₂O₃ | NA | 0.0 |
| SIRAL 40 Al₂O₃ | 0.3 | 0.0 |
| SIRAL 40 HPV Al₂O₃ | 0.3 | 0.6 |
| SIRAL 70 HPV Al₂O₃ | 0.2 | 0.6 |

### Example 13

### Constraint Index (CI) Testing: Product Distribution

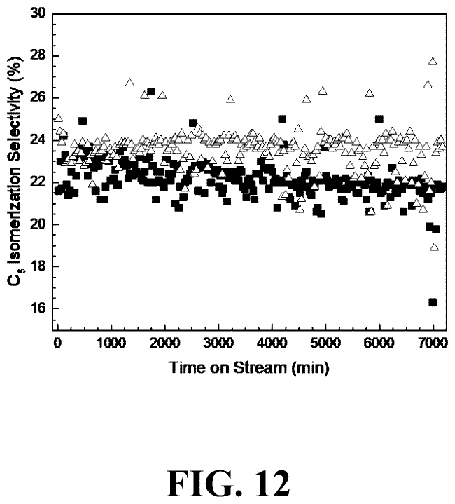

The repercussions of the highly active acid sites characterized with the present FCA catalysis system was investigated in other reactions. The performance of H1EMY (which lacks these sites as a preferential catalyst of this invention) versus conventional HMY (which contains these sites as a known catalyst of the prior art) was compared. The approach was to rely on the cracking of nC₆ and 3-MP as a probe reaction, under the same conditions as described above in Example 8. Data in FIG. 11 represent the C₆ isomerization selectivity of both catalysts. An initial C₆ isomerization selectivity was observed calculated from a linear extrapolation of initial times on stream (time on stream less than 360 minutes) in FIG. 11 to be 22.8% and 23.9% for HMY and 1EMY catalysts, respectively. At long time on streams in FIG. 12, selectivity data averaged between 6800-7200 min time on stream shows that the C₆ isomerization selectivity was 21.1% and 23.2% for HMY and H1EMY, respectively. It was concluded that under steady state conditions (long times on stream), H1EMY had a greater selectivity for isomerization compared to HMY catalysts, and furthermore, this selectivity increased with greater time on stream. In view of both H1EMY and HMY having the same conversions of alkane reactants, it was surmised that the rate of isomerization was higher for H1EMY than for HMY. It was concluded that the absence of highly active sites in the H1EMY catalyst led to lower accessible total Lewis acid sites, due to partial blockage of these sites with sodium cations, and this in turn led to higher isomerization selectivity and rates compared to the conventional catalyst known in the prior art, HMY, consisting of directly calcined and ammonium ion exchanged meso-Y. These data solidify the low-temperature FCA reaction as a relevant probe reaction that reports directly on the presence of highly active acid sites by virtue of 1,2-AMN yields above 5% under our conditions. The presence of these highly active sites results in lower isomerization yields in the CI test reaction data above.

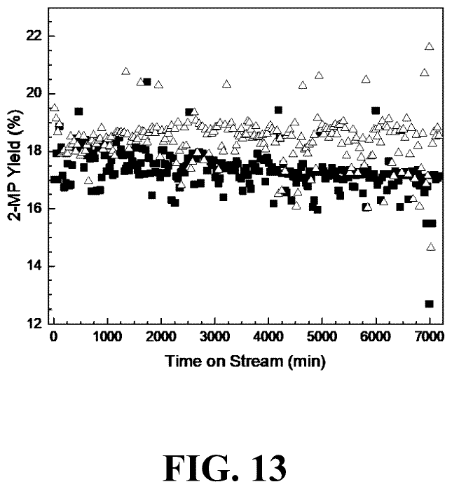

Data in FIG. 13 show the yield of 2-methylpentane (2-MP) as a function of time on stream for both EMY and HMY catalysts. An initial 2-MP yield was observed calculated from a linear extrapolation of initial times on stream (time on stream less than 380 minutes) in FIG. 13 to be 17.6% and 18.9% for HMY and H1EMY catalysts, respectively. At long time on stream as shown in FIG. 13, yield data averaged between 6800-7200 minutes time on stream demonstrates a 2-MP yield of 16.5% and 18.1% for HMY and H1EMY, respectively. These data support the discussion above regarding C₆ isomerization selectivity, which was demonstrated to be higher for H1EMY versus HMY under both initial and steady-state conditions, with this selectivity difference between the two catalysts increasing with greater time on stream.

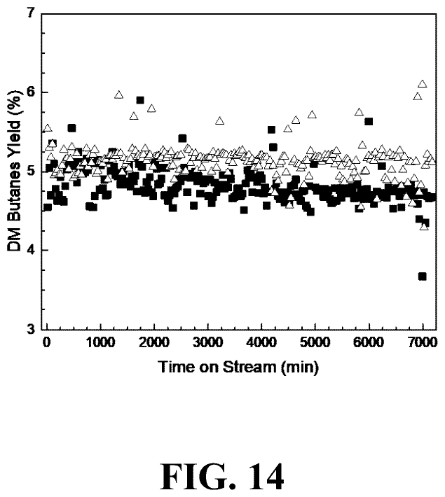

Data in FIG. 14 show the yield of dimethyl butanes (DMB) as a function of time on stream for both H1EMY and HMY catalysts. An initial DMB yield was observed calculated from a linear extrapolation of initial times on stream (time on stream less than 380 minutes) in FIG. 14 to be 4.9% and 5.3% for HMY and H1EMY catalysts, respectively. At long time on stream as shown in FIG. 14, yield data averaged between 6800-7200 minutes time on stream demonstrated a DMB yield of 4.6% and 5.1% for HMY and H1EMY, respectively. These data were consistent with the discussion above regarding C₆ isomerization selectivity, which was demonstrated to be higher for H1EMY compared with HMY under both initial and steady-state conditions, with this selectivity difference between the two catalysts increasing with greater time on stream.

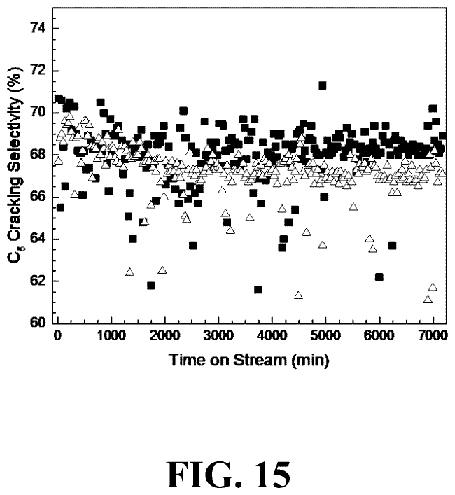

FIG. 15 shows the C₅ cracking selectivity as a function of stream for both H1EMY and HMY catalysts. Performing linear extrapolations of initial times on stream (time on stream less than 360 minutes), initial C₅ cracking selectivities of 68.7% and 68.9% for HMY and 1EMY, respectively were observed. It was concluded that at initial time on stream, H1EMY had a similar selectivity relative to HMY. At long times on stream corresponding to between 6800-7200 minutes, averaged selectivity data within this time window demonstrated C₅ cracking selectivities of 68.6% and 66.4% for HMY and H1EMY. It was concluded that H1EMY has significantly lower C₅ cracking selectivity versus HMY at longer times on stream during CI test reactions. In view of these two materials exhibiting similar nC6 and 3MP reactant conversions as a function of time on stream, it was concluded that the observed differences in C₅ cracking selectivity under steady-state conditions reflect the greater rate of C₅ cracking in HMY versus H1EMY.

### Example 14

### Constraint Index (CI) Testing: Final Coke Content

The final amount of coke on all spent catalysts was measured after CI test reactions (for tests lasting both 360 and 7200 min). This was performed by carefully removing all catalyst from the reactor tube, with particular attention on avoiding contamination with alundum in the tube. The removed catalysts were ground in a mortar and pestle to a fine powder. They were subsequently analyzed via TGA in dry air, by investigating the weight loss between 150° C. (following a 3 h soak at 150° C.) at 750° C. and comparing this weight loss for a used and fresh catalyst of the same type (on a bone dry zeolite material). These data are summarized in Table 7.

TABLE 7
Coke content deposited on various catalysts following Constraint
Index (CI) test at short time-on-stream of 360 min (STOS)
and long time-on-stream at 7200 min (LTOS).

| | | Coke |
|---|---|---|
| Sample | STOS/LTOS | Wt. % |
| CBV 720 | STOS | 4.1% |
| HMY | STOS | 2.5% |
| H1EMY | STOS | 1.1% |
| H2EMY | STOS | 2.1% |
| HECEC | STOS | 2.2% |
| HMY | LTOS | 5.4% |
| H1EMY | LTOS | 4.1% |

It was observed that for the short time-on-stream CI test experiments, the amount of coke measured on HMY and H1EMY was 2.5 wt. % and 1.1 wt. %, respectively. Increased coke corresponding to 2.2 wt. % for HECEC with the same procedure was also observed. It was surmised that catalysts with higher total Lewis acidity as a result of sodium cation unblocking and higher FCA catalytic activity under our conditions also have larger amounts of coke following the short-term CI test reactions.

Also investigated was coke for longer time on stream (7200 min) CI tests, again comparing HMY, which had a measured coke amount of 5.4 wt. %, and H1EMY had measured coke amount of 4.1 wt. %. These data further support the trends described above for coke deposits with the short time-on-stream CI tests, in which a higher degree of unblocked (by sodium cation extraction) of Lewis acidity correlated with a higher amount of measured coke in the used catalysts. The same mechanism was invoked of sodium cations selectively poisoning residual highly active Lewis acidic centers to explain the lower coke observed in H1EMY versus HMY.

The effects above consisting of higher nC₆ isomerization selectivity and rate, higher 2MP yield, higher DMB yield, lower C₅ cracking selectivity and rate, and lower coke deposits on used catalysts all demonstrate the advantages of the catalyst resulting from this invention H1EMY compared with conventional catalyst HMY. These are the direct result of the lack of presence of highly active acid sites in H1EMY as a result of sodium blockage of residual Lewis-acid sites. These highly active acid sites are present in HMY. The data above emphasize the surprising result that not all Lewis-acid sites are the same, and the small residual of Lewis acid sites that remain blocked by sodium cations in H1EMY are the ones that contribute undesired catalytic consequences above in CI test reactions and the onset of low-temperature FCA activity under our conditions. The results above further underscore the reliability of the low-temperature FCA probe reaction as a relevant probe reaction for the presence of highly active acid catalytic sites, which lead to undesirable catalytic consequences.

In conclusion, the present results demonstrated that the adsorption of sodium cations in meso-Y materials occurs in highly specific sites that adsorb sodium selectively, in a non-competitive fashion relative to other cationic species such as CTA. The results further demonstrated a high degree of sodium adsorption-site heterogeneity, with some sites having high affinity to sodium, which are extracted only after repetitive extractions, in equilibrium with low concentrations of sodium in solution, while other sites have lower affinity to sodium. The preferred catalyst resulting from this invention, H1EMY, has sufficient sodium content so as to block the sites with the highest affinity to sodium, which remain blocked even after its calcination. The results above demonstrated these sites comprise a residual amount of Lewis acid sites, which when unblocked by removing the residual sodium via its extraction contribute to low-temperature FCA activity under the present conditions. This unblocking synthesizes highly active acid sites that also contribute to undesired outcomes during CI test reactions such as lower isomerization yields, lower 2MP yields, lower DMB yields, higher cracking, and higher amounts of coke. While the preferred material of this invention H1EMY avoids these outcomes as a result of preferential blocking of residual highly active acid sites with sodium cations, its Brønsted acidity is nearly comparable to the conventional directly calcined material HMY, as ascertained by both pyridine IR spectroscopy and H/D exchange IR spectroscopy, as well as the overall conversion in CI test reactions. Thus, an advantage of the present process is it effectively removes occluded organic surfactant while maintaining a portion of active sites blocked. As mentioned above, a further advantage of 1EMY is that a significant fraction (e.g., 53%) of the CTA in ASMY can be extracted for recycling and reuse during the single-stage extraction of ASMY in MAN when synthesizing H1EMY. While extraction methods for ASMY in the prior art accomplish this last aspect of recycling and reuse of H1EMY, when synthesizing REMY, they are unable to do so while also preferentially poisoning the residual highly active sites, as H1EMY accomplishes.

As used in this disclosure the word “comprises” or “comprising” is intended as an open-ended transition meaning the inclusion of the named elements, but not necessarily excluding other unnamed elements. The phrase “consists essentially of” or “consisting essentially of” is intended to mean the exclusion of other elements of any essential significance to the composition. The phrase “consisting of” or “consists of” is intended as a transition meaning the exclusion of all but the recited elements with the exception of only minor traces of impurities.

All patents and publications referenced herein are hereby incorporated by reference to the extent not inconsistent herewith. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present process and system, but they are not essential to its practice. It is therefore to be understood that the invention may be practiced otherwise than as specifically described without actually departing from the spirit and scope of the present invention as defined by the appended claims.

While various embodiments have been described for purposes of this disclosure, various change and modifications may be made which are well within the scope contemplated by the present disclosure. Numerous other changes may be made which will readily suggest themselves to those skilled in the art and which are encompassed in the spirit of the disclosure.

What is claimed is:

1. A process for extracting an organic surfactant in an as-synthesized meso-Y zeolite (ASMY) comprising:
(a) providing an as-synthesized meso-Y zeolite (ASMY) that has been treated with an organic surfactant;
(b) treating the ASMY of (a) with methanolic ammonium nitrate (MAN); and
(c) recovering of the ASMY treated with MAN.

2. The process of claim 1, wherein the length of time of the treatment in (b) ranges from about 1 to 4 hours.

3. The process of claim 1, wherein the length of time of the treatment in (b) ranges from about 2 to 4 hours.

4. The process of claim 1, wherein the length of time of the treatment in (b) ranges from about 1.5 to 2.5 hours.

5. The process of claim 1, wherein the length of time of the treatment in (b) is about 2 hours.

6. The process of claim 1, wherein the treatment in (b) is conducted at a temperature in the range of from about 50° C. to about 80° C.

7. The process of claim 1, wherein the treatment in (b) is conducted at a temperature in the range of from about 55° C. to about 65° C.

8. The process of claim 1, wherein the treatment in (b) is conducted at a temperature in the range of from about 60° C.

9. The process of claim 1, wherein the methanolic ammonium nitrate used in the treatment of (b) comprises about 25 to 100 mM (millimoles) of ammonium nitrate in methanol.

10. The process of claim 1, wherein the methanolic ammonium nitrate used in the treatment of (b) comprises about 50 to 90 mM of ammonium nitrate in methanol.

11. The process of claim 1, wherein the methanolic ammonium nitrate used in the treatment of (b) comprises about 70 to 80 mM of ammonium nitrate in methanol.

12. The process of claim 1, wherein the methanolic ammonium nitrate used in the treatment of (b) comprises about 75 mM of ammonium nitrate in methanol.

13. The process of claim 1, wherein the ratio of mgs (milligrams) of ASMY to ml. of MAN used in the treatment of (b) ranges from about 50:1 to 10:1.

14. The process of claim 1, wherein the ratio of mgs of ASMY to ml. of MAN used in the treatment of (b) ranges from about 40:1 to 15:1.

15. The process of claim 1, wherein the ratio of mgs of ASMY to ml. of MAN used in the treatment of (b) ranges from about 30:1 to 20:1.

16. The process of claim 1, wherein the ratio of mgs of ASMY to ml. of MAN used in the treatment of (b) is about 20:1.

17. The process of claim 1, wherein the ASMY treated with MAN recovered in (c) comprises 18 wt % or less of the organic surfactant.

18. The process of claim 1, wherein the ASMY treated with MAN recovered in (c) comprises 15 wt % or less of the organic surfactant.

19. The process of claim 1, comprising a further extraction achieved by repeating steps (b) and (c) at least one more time.

20. The process of claim 1, comprising a further extraction achieved by repeating steps (b) and (c).

21. The process of claim 1, wherein the length of time of the treatment in (b) ranges from about 1 to 4 hours; the treatment in (b) is conducted at a temperature in the range of from about 50° C. to about 80° C.; the MAN used in the treatment of (b) comprises from about 25 to 100 mM of ammonium nitrate in methanol and the ratio of mgs of ASMY to ml of MAN in the treatment of (b) ranges from about 50:1 to 10:1.

22. The process of claim 1, wherein the length of time of the treatment in (b) ranges from about 1.5 to about 2.5 hours; the treatment in (b) is conducted at a temperature in the range of from about 55° C. to about 65° C.; the MAN used in the treatment of (b) comprises from 70 to 80 mM of ammonium nitrate in methanol, and the ratio of mgs of ASMY to ml of MAN in the treatment of (b) ranges from about 30:1 to about 20:1.

23. The process of claim 21, wherein the ASMY treated with MAN recovered in (c) comprises 18 wt % or less of the organic surfactant.

24. The process of claim 22, wherein the ASMY treated with MAN recovered in (c) comprises 18 wt % or less of the organic surfactant.

25. The process of claim 21, wherein the ASMY treated with MAN recovered in (c) comprises 15 wt % or less of the organic surfactant.

26. The process of claim 22, wherein the ASMY treated with MAN recovered in (c) comprises 15 wt % or less of the organic surfactant.

27. A process for preparing a catalyst which comprises calcining the ASMY treated with MAN recovered in claim 1.

28. A process for preparing a catalyst which comprises calcining the ASMY treated with MAN recovered in claim 17.

29. A process for preparing a catalyst which comprises calcining the ASMY treated with MAN recovered in claim 18.

30. A process for preparing a catalyst which comprises calcining the ASMY treated with MAN recovered in claim 19.

31. A process for preparing a catalyst which comprises calcining the ASMY treated with MAN recovered in claim 20.

32. The process of claim 1, wherein the organic surfactant comprises a cetyltrimethylammonium surfactant.

33. An ASMY treated with MAN prepared by the process of claim 1.

34. An ASMY treated with MAN prepared by the process of claim 21.

35. An ASMY treated with MAN prepared by the process of claim 22.

36. A catalyst prepared by the process of claim 27, which comprises a portion of highly active sites masked by sodium cations.

37. A catalyst prepared by the process of claim 29, which comprises a portion of highly active sites masked by sodium cations.

38. A catalyst prepared by the process of claim 31, which comprises a portion of highly active sites masked by sodium cations.

39. A cracking process comprising reacting hydrocarbons over the catalyst of claim 36.

40. A Friedel-Crafts acylation reaction comprising reacting hydrocarbons over the catalyst of claim 36.
